(19) United States
(12) Patent Application Publication
(10) Pub. No.: US 2025/0107843 A1
(43) Pub. Date: Apr. 3, 2025

(54) CATHETER SYSTEM FOR THE TREATMENT OF ATRIAL FIBRILLATION

(71) Applicant: ELECTROPHYSIOLOGY FRONTIERS S.P.A, Torino (IT)

(72) Inventors: Marwan ABBOUD, Pierrefonds (CA); Douglas WAHNSCHAFFE, Otsego, MN (US)

(21) Appl. No.: 18/780,754

(22) Filed: Jul. 23, 2024

Related U.S. Application Data
(63) Continuation of application No. 18/517,297, filed on Nov. 22, 2023, now abandoned.

(30) Foreign Application Priority Data
Oct. 2, 2023 (IT) 102023000020334

(51) Int. Cl. A61B 18/14; A61B 18/00

(52) U.S. Cl. CPC A61B 18/1492; A61B 2018/00267; A61B 2018/00279; A61B 2018/00375; A61B 2018/00577

(57) ABSTRACT

An anchored cardiac ablation uses a catheter having an ablator head having ablator elements, or petals and an anchor. The catheter is advanced in the PV and expand the anchor in the PV; the ablation head is opened once in the PV, to reach the PV walls; the ablation element is pulled back in operating position, keeping the anchor in place. When the ablation head reaches the region of the vein ostium, the diameter of the ablation elements increase, and the user stops pulling back. The ablation element is moved toward the ostium, positioning the ablation elements on the tissue to be treated; desired target tissue is ablated; the ablator and anchor are collapsed into resting positions and withdrawn from the PV.

### CROSS-REFERENCE TO RELATED APPLICATION

This application is a continuation of U.S. application Ser. No. 18/517,297, filed Nov. 22, 2023, and claims the benefit of Italian Application Serial No. 102023000020334 file Oct. 2, 2023, the entirety of which are incorporated by reference herein.

### FIELD

The present invention relates generally to atrial fibrillation, and specifically to treating the atrial fibrillation by pulmonary vein isolation. Catheter and ablation apparatuses for human tissue ablation are described.

### BACKGROUND

The human heart has four chambers. The two upper chambers are the left and right atrium, and the two lower chambers are the left and right ventricles. Blood from the veins of the body returns to the right atrium of the heart. When the right atrium contracts, the blood passes from the right atrium through the tricuspid valve to the right ventricle. The blood is then pumped by contraction of the right ventricle through the pulmonary artery to the lungs. In the lungs, carbon dioxide passes out of the blood, and oxygen passes into the blood. The oxygenated blood returns from the lungs through the pulmonary veins to the left atrium. The blood is pumped by contraction of the left atrium through the mitral valve to the left ventricle. Contraction of the left ventricle pumps the blood out of the left ventricle to the aorta and through the arteries to the body.

The contraction of heart muscle occurs in response to electrical impulses which trigger fibers of heart muscle to contract in a coordinated fashion. During sinus rhythm, the heartbeat starts in the right atrium with an electrical impulse at the sinoatrial (SA) node. The impulse spreads through the right and left atrium and then to the atrio-ventricular (AV) node. The AV node is an electrical pathway that transmits electrical signals from the atria to the ventricles. The electrical signal travels from the AV node along a common pathway and then splits into left and right bundle branches to activate the left and right ventricles. The sequence of activation results in efficient pumping. The atria contract first, and pump blood to the ventricles. The ventricles then contract and pump blood to the lungs and the body. During sinus rhythm, the AV node permits the ventricles to beat at the same rate as the atrium, but with a slight delay which allows the atria to empty their blood into the ventricles before the ventricles contract.

Cardiac arrhythmias, such as atrial fibrillation (also referred to as “AF” or “afib”), occur when regions of cardiac tissue abnormally conduct electric signals to adjacent tissue. This disrupts the normal cardiac cycle and causes asynchronous rhythm. For example, during AF, the atrial muscle activates at rates that can exceed 300 beats per minute. The atria no longer pump blood efficiently to fill the ventricles. This can result in a variety of chronic and undesirable conditions.

AF is the most common sustained arrhythmia in humans. It affects anywhere from 0.4% to 1% of the general population and increases in prevalence with age to approximately 10% in patients over 80 years of age. By the year 2050, it is estimated that 1 in 5 Americans over the age of 65, i.e., 7.6 million individuals, is expected to suffer from AF. AF is a serious condition that leads to a 1.9-fold increased risk of mortality, and a 5-fold higher risk of stroke. Additionally, heart disease costs the United States about $219 billion each year.

Pulmonary vein isolation (PVI) is the cornerstone of treating AF, and it is used for Paroxysmal AF (PAF), Persistent AF (PsAF) and Permanent AF. However, while the results of PVI for patients with PAF are consistently good, the results for patients with PsAF and Permanent AF are much more variable. Clinical data is showing that in the case of Persistent and Permanent AF treatment starts with isolation of pulmonary veins similar to the treatment of Paroxysmal AF but extra ablation of other areas of the heart are needed. As an example, Posterior Wall Isolation (PWI) is a target for Persistent AF (PsAF) and Permanent AF.

Notwithstanding the open debate, PVI remains the first-line treatment for AF, wherein the primary clinical benefit of AF ablation is improvement in quality of life resulting from the elimination of arrhythmia-related symptoms such as palpitations, fatigue, or effort intolerance.

In today's clinical state-of-the-art, several problems remain unresolved in PVI.

AF ablation can be performed from the inside of the heart via catheters that are introduced percutaneously from the veins in the groin or neck. Alternatively, it can be accomplished from the outside of the heart with either open heart surgery or via a thoracoscopic approach. A mixed or hybrid approach is also available. The most common approach is the catheter-based approach. This is considered a minimally invasive procedure as no surgical incisions are required. Catheter-based ablation techniques destroy the tissue which is producing the unwanted electrical activation or destroy conductive pathways to electrically isolate the pulmonary vein from the left atrium.

To accomplish this, an access to the left atrium is done through a standard transeptal sheath to cross the septum from the right atrium into the left atrium. Once the transeptal puncture is done, a guide wire is inserted and the transseptal sheath is replaced by a deflectable sheath and positioned inside the left atrium. Prior to insertion of the catheter inside the deflectable sheath, all catheter open ports are flushed with heparinized saline solution to ensure that no air is released inside the heart chamber during insertion of the device. For this purpose, the catheter has more than one port allowing the flushing of all open lumens with saline that could be in contact with blood.

Initially, focal trigger elimination was performed within the PVs at the site of earliest activation. This concept of “focal ablation” has been largely abandoned due to a low long-term success rate, the considerable risk of PV stenosis, and the lack of a clearly defined procedural endpoint.

Two alternate ablation strategies have been developed: (i) segmental ostial PVI and (ii) circumferential PV ablation (CPVA). Segmental ostial PVI is an electrophysiologically guided technique aimed at electrical disconnection of the PVs at the level of the PV ostium. PVI is achieved by sequential RF delivery. With this technique, approximately 50% of the ostial circumference is targeted (Oral H et al., Segmental ostial ablation to isolate the pulmonary veins during atrial fibrillation: feasibility and mechanistic insights. Circulation. 2002; 106:1256-62). CPVA, initially described by Pappone et al. (Pappone C et al., Circumferential radiofrequency ablation of pulmonary vein ostia: A new anatomic approach for curing atrial fibrillation. Circulation. 2000; 102:2619-28), is an anatomical approach to encircle the PVs by ablating on the atrial aspect of the LA-PV junction under the guidance of a non-fluoroscopic 3-dimensional electro anatomical mapping system. Ablation line continuity was originally defined by voltage abatement within the encircled areas and a pre-defined activation delay between contiguous points lying in the same axial plane inside and outside the ablation line. This approach by design does not involve verification of PVI, and it could be demonstrated that only 55% of PVs were isolated after CPVA. Subsequently, Ouyang et al. (Ouyang F et al., Recovered pulmonary vein conduction as a dominant factor for recurrent atrial tachyarrhythmias after complete circular isolation of the pulmonary veins: lessons from double Lasso technique. Circulation. 2005; 111:127-35) demonstrated the feasibility of complete isolation of the PVs with continuous circular lesions placed around the ipsilateral PV pairs guided by the double-Lasso technique and 3-dimensional mapping.

The recognition that the PV antrum plays an essential role in the generation and perpetuation of AF and that targeting the tubular portion of the PV is still associated with risk of PV stenosis led to a shift of the lesion set away from the PV ostia towards the left atrium (LA) thereby including portions of the LA posterior wall, of the posterior septum, and of the LA roof (Kiuchi K et al., Quantitative analysis of isolation area and rhythm outcome in patients with paroxysmal atrial fibrillation after circumferential pulmonary vein antrum isolation using the pace-and-ablate technique. Circ Arrhythm Electrophysiol. 2012; 5:667-75).

Testing for entrance block can help confirm PVI, although complex electrograms that consist of both near- and far-field potentials may make assessment of entrance block challenging. Differential pacing manoeuvres can help appropriately identify PV potentials. After entrance block has been achieved, pacing within the PVs to demonstrate capture of PV musculature with exit block may also help to confirm completeness of lesion sets for PVI. Exit conduction assessment allows for a clear distinction to be made between near- and far-field signals that become dissociated when exit block occurs. Further, exit conduction is easily recognized, if existent, because of capture of the atrial rhythm.

Given the limitations of EGM analysis alone for the assessment of entry and exit block to confirm that the lesion sets are complete, additional techniques have been reported. One such method involves pacing at high output using the mapping ablation catheter along the ablation line and targeting areas of capture with adjunctive ablation. The examination of EGM morphology has been proposed as a method for achieving and confirming PVI. Waveform analysis of local EGM morphology may allow for the automated confirmation of PVI status, though this method is not currently used widely.

Once the catheter is inserted inside the left atrium through the deflectable sheath, multiple challenges are faced to ensure an effective pulmonary vein isolation.

The ability of the physician to deflect and access the pulmonary vein is a challenging task mainly when dealing with right pulmonary veins. For this purpose, physician uses guide wire that is inserted inside the vein allowing the ablation catheter to track over the wire toward the vein lumen.

Once the ablation head is engaged, the second challenge is to be able to centre the ablation head around the ostium of the vein with the help of the deflection mechanism of the deflectable sheath and the guide wire. The main challenge is that the guide wire is very soft and, in most cases, does not allow to effectively centre the ablation element. A much more rigid guide element is needed to allow proper centring around the ostium of the vein.

Once the ablation head is centred, the contact between the ablation head and the desired region of ablation must have a continuous contact to prevent having electrical gaps that are not isolated. For this purpose, the ablation head must be able to reach the angle of conformability, wherein the tissue has an irregular surface and there is a need for an ablator that can conform to the 3D anatomy of the pulmonary vein-left atrial junction to isolate successfully more precisely and easily the pulmonary.

Once the ablation head is correctly positioned and centred, means are required to keep it in the correct position for the required time.

Before delivering energy, there is a need to sense the intracardiac signals from inside the vein lumen to be able to assess the isolation during and after the delivery of the ablation energy. For this purpose, an independent sensing element must be positioned away from the ablation region and to the inside lumen of vein but positioned at the muscle sleeve of the junction between the pulmonary vein and the atrium. Once the sensing element is engaged, physician move back and forth the sensing element until an intracardiac signals are detected. At this point, physician could perform pacing at this location to ensure that position is the optimal position.

Finally, the energy has to be delivered in a focussed manner, i.e., on the circumference, and not into the centre lumen of the vein, to enable the rapid isolation of the pulmonary veins with minimal collateral damages to adjacent structures.

The main ablation energies that are commonly used to tissue ablation are radio frequency (RF), cryoablation and pulsed field ablation (PFA).

The goal of RF ablation is to induce thermal injury to the tissue through electromagnetic energy deposition. The term RF ablation applies to coagulation induced by all electromagnetic energy sources with frequencies less than 900 kHz, although most devices function in the range of 375-500 kHz. The term RF refers not to the emitted wave but rather to the alternating electric current that oscillates in this frequency range. The thermal damage caused by RF heating is dependent on both the tissue temperature achieved and the duration of heating. Heating of tissue at 50-55° C. for 4-6 min produces irreversible cellular damage. At temperatures between 60° C. and 100° C. near immediate protein coagulation is induced, with irreversible damage to mitochondrial and cytosolic enzymes as well as nucleic acid-histone protein complexes. Cells experiencing this extent of thermal damage most often, but not always, undergo coagulative necrosis over the course of several days.

Cryoablation refers to all methods of destroying tissue by freezing. Cryoablation causes cellular damage, death, and necrosis of tissues by direct mechanisms, which cause cold-induced injury to cells, and indirect mechanisms, which cause changes to the cellular microenvironment and impair tissue viability. Cellular injury, both indirect and direct, can be influenced by four factors: cooling rate, target temperature, time at target temperature, and thawing rate.

PFA employs trains of high voltage in very short duration electrical pulses to injure tissue by the mechanism of irreversible electroporation. This approach is nonthermal, and myocardium is highly susceptible to this type of injury, whereas collateral structures seem to be relatively resistant to injury. Thus, PFA offers the promise of achieving durable pulmonary vein ablation with very low risk. In addition, using a circular multipolar catheter, pulmonary vein isolation can be achieved very rapidly (within one minute). Accordingly, PFA may dramatically shorten the procedure time.

Devices and methods here disclosed overcome the drawbacks and the still open technical problems of the known art.

### SUMMARY

The present invention includes a catheter apparatus (1), comprising a control handpiece (12), a catheter shaft (11), an ablation head (3), a catheter tip (15), the catheter shaft (11) having an external tubular body (2) and comprising two portions: a proximal portion, closer to the control handpiece, and a distal portion, toward the catheter tip, wherein along the catheter shaft (11) a fixing point (14) is defined, wherein the fixing point (14) is in the distal portion of the catheter shaft, wherein the ablation head comprises at least two ablation elements or petals (3a), each petals (3a) comprising: a circumferential peripheral portion (3b), substantially along an arc of circumference having the longitudinal axis of the catheter shaft (11) as its centre, comprising one or more electrodes, thus forming a circumferential linear ablation array (CLA); two legs (3c), each one connected to an end of the portion (3b), in correspondence with a curved section therein, wherein one legs is a fixed leg (3d), the other one is a movable leg (3e). Two elements, a movable element (8) and a fixing element (9), are included in the catheter, the movable element (8) being movable along the catheter shaft, the fixing element (9) being connected to the catheter shaft, at the fixing point (14). Each one of the petals (3a) is connected, via the fixed leg (3d), to the fixing element (9) and, via the movable leg (3e), to the movable element (8); wherein from the control handpiece the opening and closure of the petals is controlled via the movable element (8).

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1A is a schematic view of the working setting of the catheter apparatus;

FIG. 1B is an enlarged view of the catheter apparatus, the ablation head with the anchor and the control handpiece

FIG. 2A is a perspective view of the catheter in an operational embodiment;

FIG. 2B, FIG. 2C and FIG. 2D are perspective views of an ablator and anchor in three steps of the procedure;

FIG. 3A is a perspective view of the ablator and the anchor, in an illustrative embodiment, when the ablator is in resting position;

FIG. 3B is a perspective view of the ablator and the anchor, in an illustrative embodiment, when the ablator is in operating position;

FIG. 4A, FIG. 4B, FIG. 4D and FIG. 4E are cross sectional views of a portion of the catheter, showing control means controlling the opening and closing of the CLA, in four different embodiments;

FIG. 4C illustrate a top view of the fixing element 9;

FIG. 4F illustrate a top view of the movable element 8;

FIG. 4G is a cross sectional view of a portion of the catheter, showing control means controlling the CLA, in an embodiment comprising an anchor, too;

FIG. 5A illustrates a side view of the petals in operating position;

FIG. 5B, FIG. 5C, FIG. 5D and FIG. 5E illustrate a frontal view of the petals in operating position in four different embodiments;

FIG. 5F illustrates the ablation element in operating position;

FIG. 5G illustrates the portion of the catheter apparatus comprising the ablation head and the anchor, in resting position, and a magnification of a petal, showing the bending;

FIG. 5H and FIG. 5I illustrate two embodiments of petal's bending;

FIG. 6A and FIG. 6B illustrate punctiform ablation electrodes, in two possible configurations;

FIG. 6C illustrates two cross sectional view of electrodes;

FIG. 6D is an exemplificative scheme of a possible embodiment of punctiform electrodes on an ablation element comprising four petals;

FIG. 6E is an exemplificative drawing of linear electrodes on the petals;

FIG. 6F is a picture illustrating two exemplificative laser cut linear electrodes;

FIG. 7 is a schematic drawing of an embodiment of electrode insulation;

FIG. 8A and FIG. 8B illustrate a perspective view of the anchor and the ablation head in stamping position;

FIG. 9 illustrates an embodiment wherein the guide wire shaft is flexible; and

FIG. 10 illustrates a portion of the catheter apparatus comprising flushing ports.

### DETAILED DESCRIPTION

The invention is described hereunder with reference to non-limiting examples, provided for illustrative and non-limiting purposes in the enclosed drawings. The drawings illustrate different aspects and embodiments of the present invention and, when appropriate, reference numbers illustrating structures, components, materials and/or similar elements in different figures are indicated with similar reference numbers.

It should be understood, however, that there is no intention of limiting the invention to the specific embodiment illustrated but, on the contrary, the invention intends to cover all the modifications, alternative constructions, and equivalents that fall within the scope of the invention as defined in the claims.

The use of “for example”, “etc.”, “or” indicates non-exclusive alternatives, without limitation, unless otherwise specified. The use of “comprises” means “comprises, but not limited to” unless otherwise specified.

In general, the term “ablation” refers, in the medical field, to the treatment of a tissue suitable for removing a surface part of the same tissue or necrotizing it and/or causing a cicatrisation of the same.

The ablation referred to in this invention is specifically destined for interrupting the electric continuity of the tissue in correspondence with the zone treated by ablation.

In this sense, ablation can take place with a series of treatments, for example by means of an electric current, by heat, cryogenics, RF, PFA or other forms of treatment.

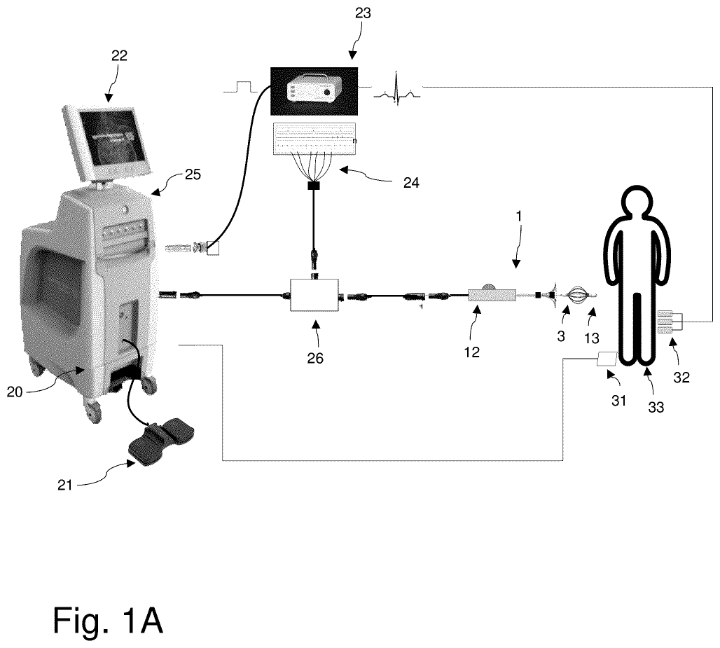

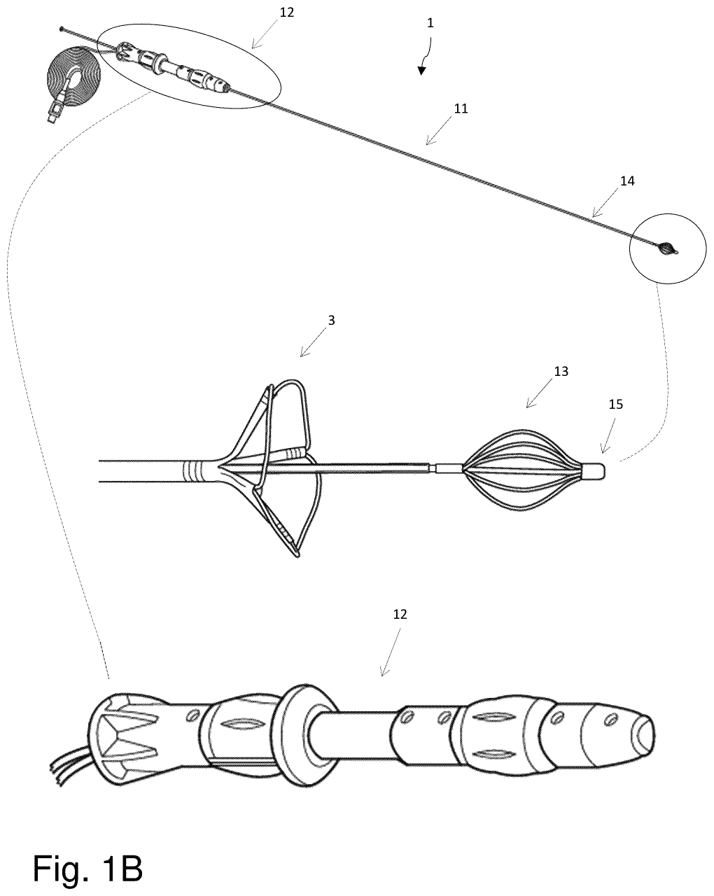

This invention is for a catheter apparatus used in the treatment of heart arrhythmia, preferably AF, the catheter apparatus 1, with reference to FIG. 1B, comprising a control handpiece 12, a catheter shaft 11, an ablation head 3, an anchor 13 and a catheter tip 15.

The catheter shaft 11 comprises two portions: a proximal portion, closer to the control handpiece, and a distal portion, toward the tip. Along the catheter shaft 11 a fixing point 14 is defined, wherein the fixing point 14 is in the distal portion. The ablation head 3 and the anchor 13 are along the distal portion of the catheter shaft, which terminates with the catheter tip 15.

With reference to FIG. 1A, the catheter apparatus 1 is conveniently connected through an interface 26 to an interface unit 25 that typically comprises electronic/software subsystems that controls the connection and/or disconnection or activation of catheter electrodes or other electrical components prior, during and post energy delivery. The interface unit is equipped with a graphical user interface 22. A foot switch 21 is conveniently adopted to allow the user to interact with the interface unit. The interface unit allows the connection between the catheter and electrophysiology (EP) mapping and/or EP recording systems. A generator 20 is required, as an energy source. The catheter apparatus reaches the subject to be treated 33, and the activity is controlled with surface ECG electrodes 32, whose signals is collected by an ECG processing unit 23, transmitting the elaborated signal to the interface unit 25. An ECG interface box 24 provide a connection interface to connect the ECG collected data from the catheter electrodes to the EP lab equipment. A dispersive pad 31 is conveniently in contact with the subject and the generator.

The control handpiece 12 remains external once the catheter apparatus is inserted into the body of the subject to be treated. The operator uses the control handpiece for controlling the action of the catheter itself, being operatively connected to the anchor and the ablation head. The “operative connection” can be actuated in numerous ways, for example by control means and electrical wire.

The catheter tip 15 reaches the final destination in the heart of the subject to be treated.

FIG. 1B shows a magnification of the catheter apparatus 1, with the control handpiece 12 at one end and the ablation head 3 and the anchor 13 at the opposite one.

The form of the control handpiece is of no particular interest for the present invention, as it is produced analogously to those known in the art; consequently, no further detail is provided herein with respect to the handpiece.

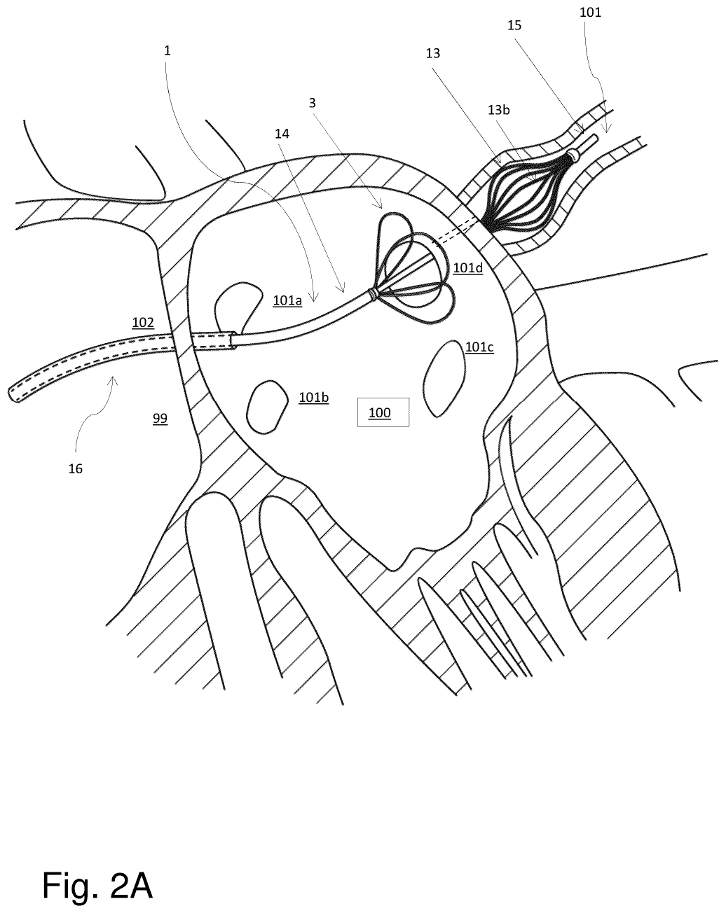

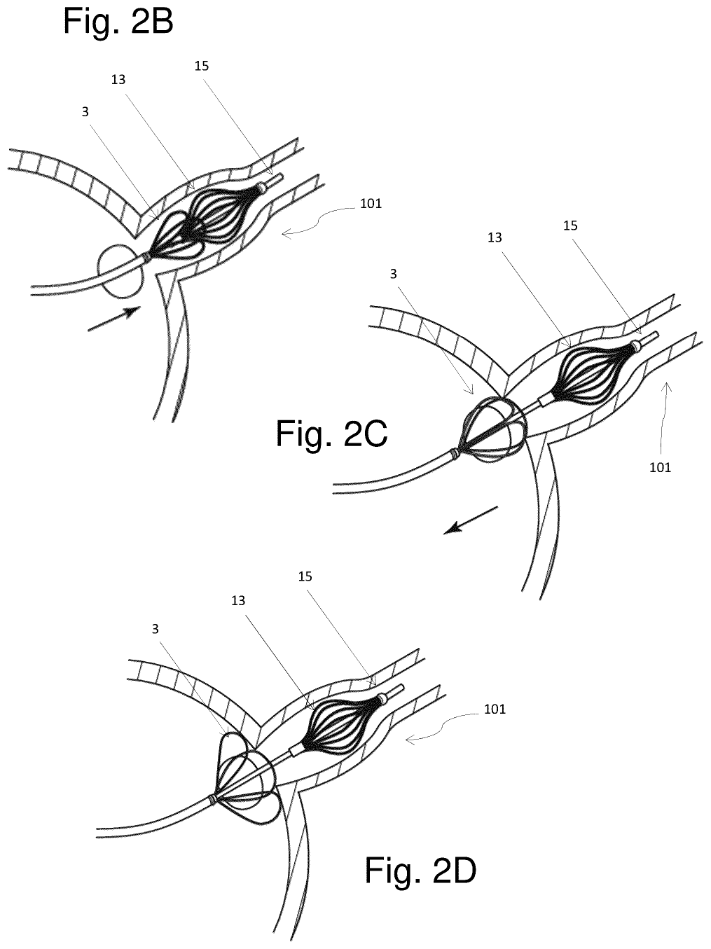

In general terms, and with reference to FIG. 2A, showing the catheter apparatus in an operational embodiment, the access to the left atrium 100 is done through a standard transeptal sheath to cross the septum 102. Once the transeptal puncture is done, a deflectable sheath 16 is inserted and positioned inside the left atrium. Prior to insertion of the catheter inside the deflectable sheath, all catheter open ports are flushed with heparinized saline solution to ensure that no air is released inside the heart chamber during insertion of the device. For this purpose, the catheter has more than one port allowing the flushing of all open lumens that could be in contact with blood, as described below in further details.

In one embodiment, the catheter 1 reaches the left atrium 100 through the deflectable sheath 16. Once inserted into the pulmonary vein 101, the anchor 13 is deployed. The anchor extends until a radial wall force is reached. Although an anchor 13 and umbrella ablation head 3 are shown in these operational embodiments, any anchoring technique and ablating device as contemplated herein can be employed.

In the embodiment depicted in FIG. 2A, the pulmonary vein ostium 101d is the treated one, the more distal and easier to reach from the catheter advancing from the septum 102. When the treatment is required in one of the other pulmonary veins, for example at the right superior one, the movement required is more complex.

The anchor enters into the vein, extends to reach the vein wall, allowing to secure the anchor in position. The anchor shaft 13b is secured to the anchor and it is centered, thus allowing the ablation head to be tracked over the anchor shaft to reach the proper position around the PV. In this way, the ablation head is as centered as possible into the PV, touching the tissue to be ablated.

In an embodiment, according to FIGS. 2B, C, D once the anchor is positioned inside the PV, the ablation element enters the PV, too. Once in the PV, the ablation element is opened until a certain diameter to reach the PV walls (FIG. 2B). Then, the ablation element is pulled back, away from the anchor which is kept in place. The diameter of the ablation element would suddenly increase once it reaches the region of the vein ostium (FIG. 2C). User will stop at this position, push back the ablation element toward the ostium, therefore reaching an optimal positioning (FIG. 2D) of the ablation element on the tissue to be treated, with sufficient contact force with the tissue.

It is here described a method or procedure for performing an anchored cardiac ablation, comprising:
Making available a catheter comprising an ablator head comprising ablator elements and an anchor, wherein the ablator head has an operating position and a resting position, wherein, in the operating position, the ablation elements extends and broaden out both radially and axially towards the anchor; wherein the anchor has an operating position and a resting position, wherein, in the operating position, anchor arms protrude radially from the catheter shaft;
Advancing the catheter into PV and extending anchor arms into the PV, positioning the anchor inside the PV;
Opening the ablation head once in the PV, to reach the PV walls;
Pulling back the ablation element in operating position, keeping the anchor in place;
When the ablation head reaches the region of the vein ostium, the diameter of the ablation element increase and user stop pulling back;
Pushing back the ablation element toward the ostium, reaching the desired positioning of the ablation element on the tissue to be treated;
Initiating ablation energy to commence ablation of the desired target tissue;
Once ablation is complete, collapsing the ablator and anchor into resting positions; and Withdrawing the catheter out of the PV.

In another embodiment, the method or procedure for performing an anchored cardiac ablation comprises:
Making available a catheter comprising an ablator head comprising ablator elements and an anchor, wherein the ablator head has an operating position and a resting position, wherein, in the operating position, the ablation elements extends and broadens out both radially and axially towards the anchor; wherein the anchor has an operating position and a resting position, wherein, in the operating position, anchor arms protrude radially from the catheter shaft;
Advancing the catheter into PV and extending anchor arms into the PV, positioning the anchor inside the PV;
Opening the ablation head inside the atrium chamber;
Pushing forward the ablation element in operating position, keeping the anchor in place;
When the ablation head reaches the region of the vein ostium, initiating ablation energy to commence ablation of the desired target tissue;
Once ablation is complete, collapsing the ablator and anchor into resting positions; and
Withdrawing the catheter out of the PV.

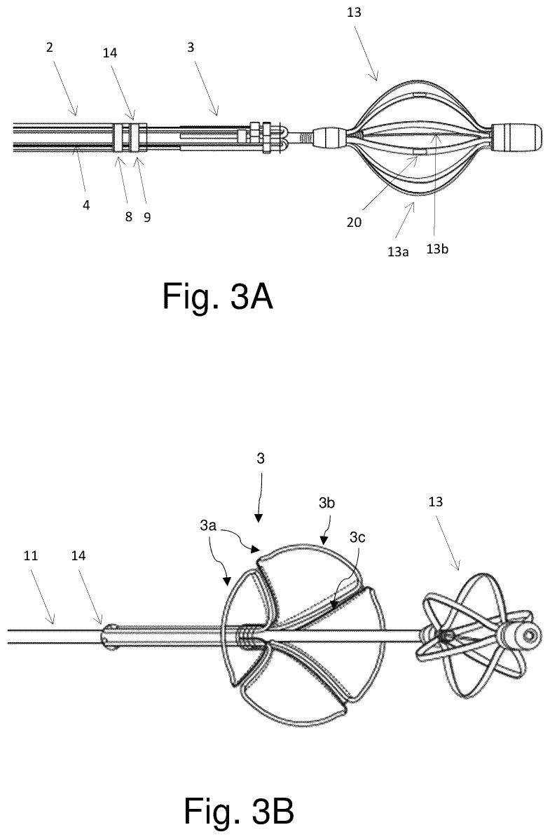

With reference to FIG. 3A, B, the ablation head 3 comprises at least two ablation elements or petals 3a that can be moved from a rest position, FIG. 3A, in which they are housed in the external tubular body 2 of the catheter shaft 11, and an operating position in which they protrude from the external tubular body 2 like a petal, FIG. 3B.

The movement between the two positions, rest and operating, is actuated thanks to a mechanical control positioned in the handpiece of the device and which allows the controlled and adjustable extraction of the petals 3a, via control means 4.

The anchor 13 comprises anchor arms, or splines 13a and an anchor shaft 13b. In an embodiment, the splines are 5 splines. In an embodiment, are 6 splines. On the splines, there are sensing electrodes 20, used to sense electrical activity on the heart muscles. Each spline could contain one or more electrodes allowing unipolar or bipolar reading of the ECG.

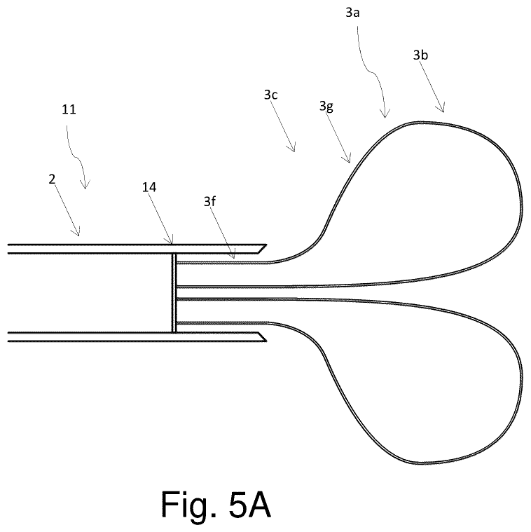

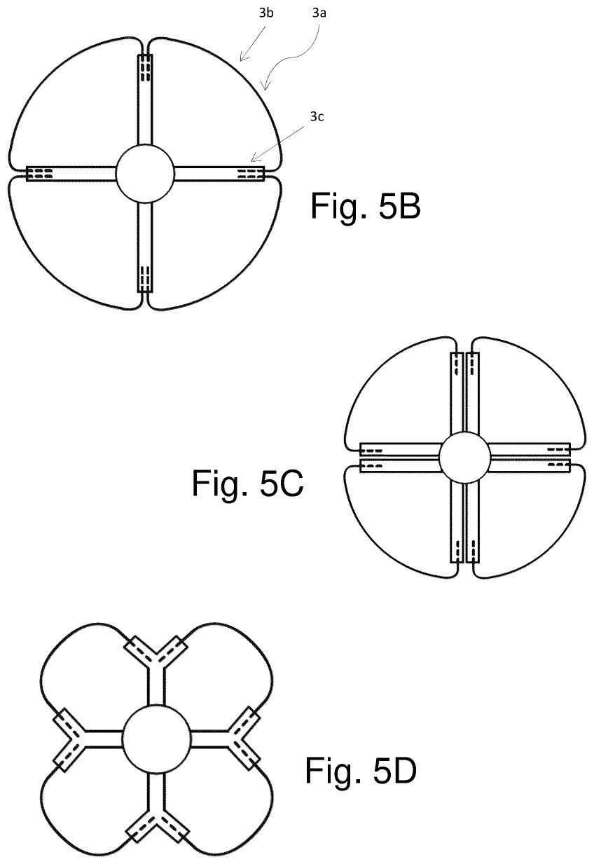

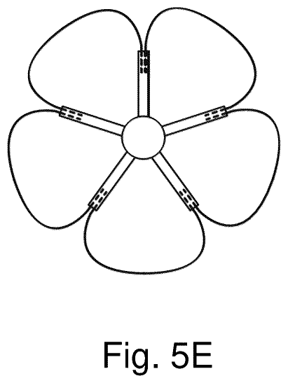

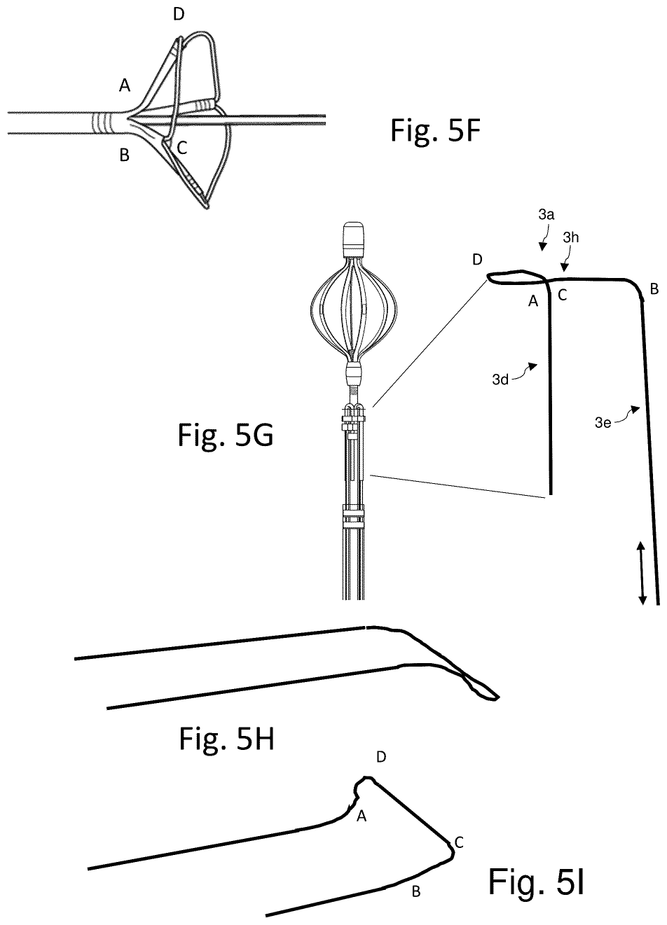

With reference to FIG. 5, each of the ablation elements or petals 3a comprises:
a circumferential peripheral portion 3b, substantially along an arc of circumference having the longitudinal axis of the catheter shaft 11 as its centre, comprising one or more electrodes, thus forming a circumferential linear ablation array (CLA);
two legs 3c, each one connected to an end of the CLA 3b, in correspondence with a curved section wherein, of these two legs 3c, one leg is a fixed leg 3e, the other one is a movable leg 3d; each ablation petal 3a being separate and distinct from another ablation petal 3a of the ablation head, all the ablation petals 3a of the ablation head 3 being separately connected to a distinct electric energy generator.

In a first embodiment, the present invention solves the problem of how to facilitate the movement of the control means controlling the opening and the closing of the petals. It is in fact clear to the person skilled in the art that, having several wires included in a tubular body which extends form a control handpiece, external to the subject to be treated, to the left atrium of the same, creating a tortuous path, with twists and turns, leads the wires to get entangled, making their handling difficult.

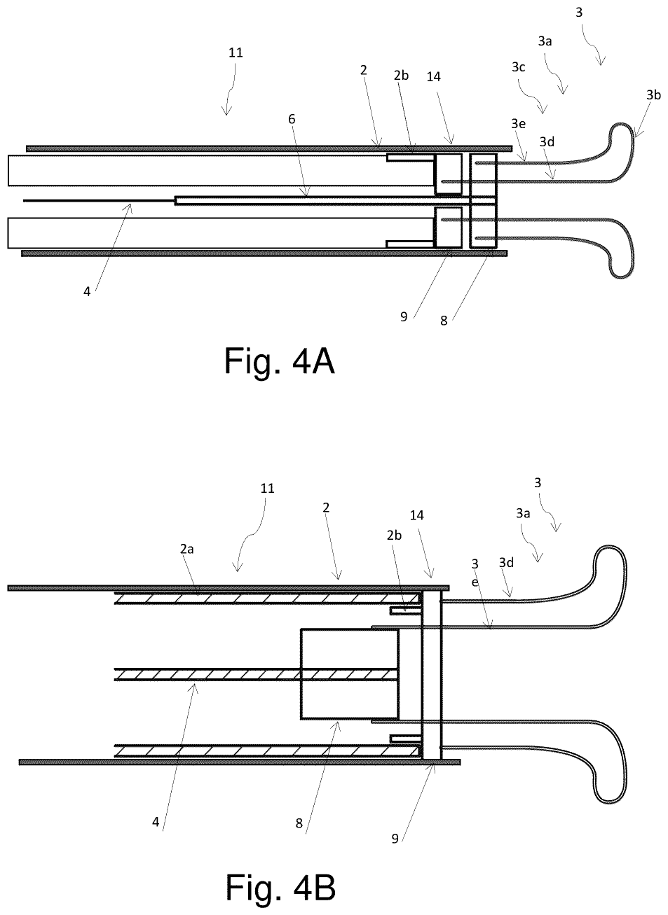

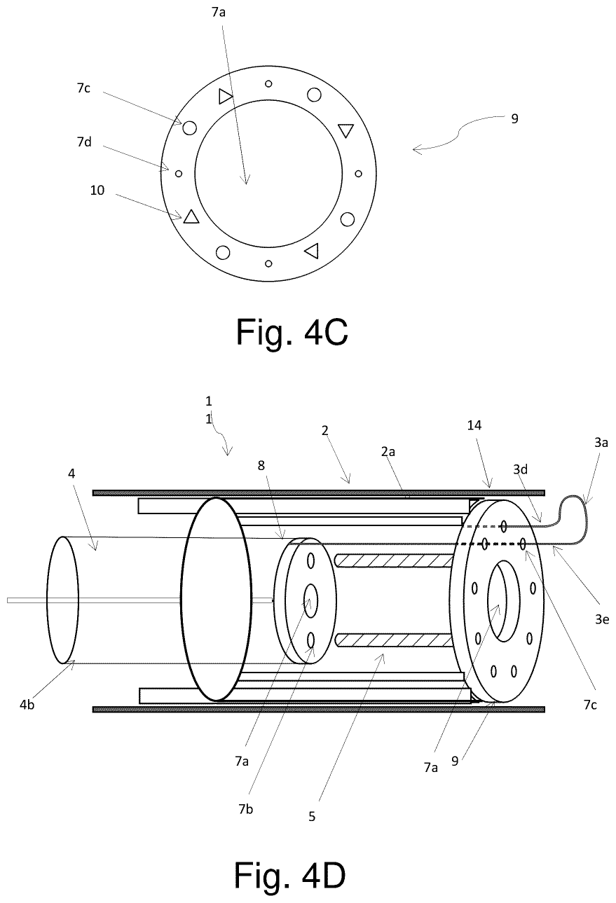

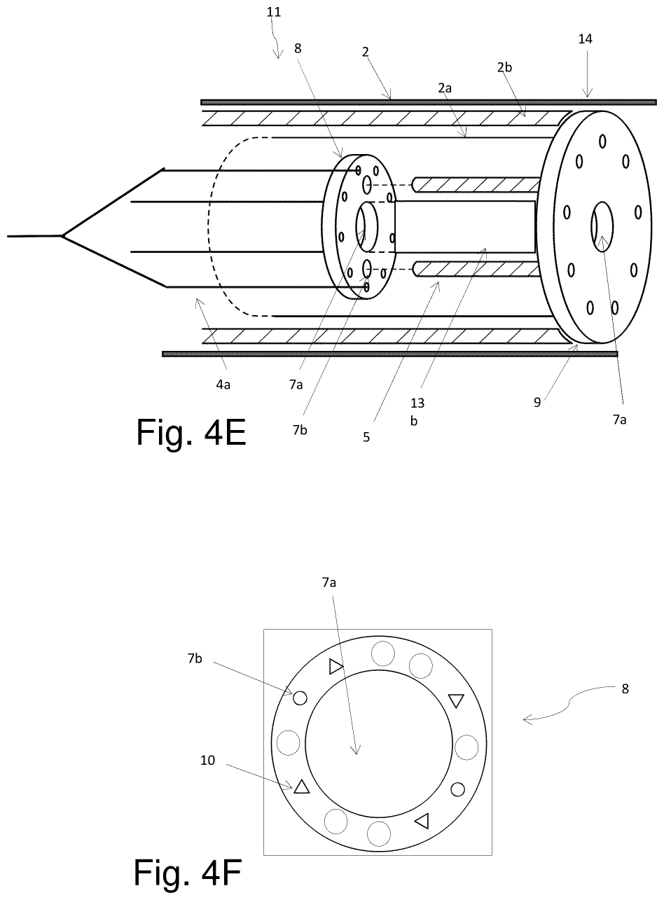

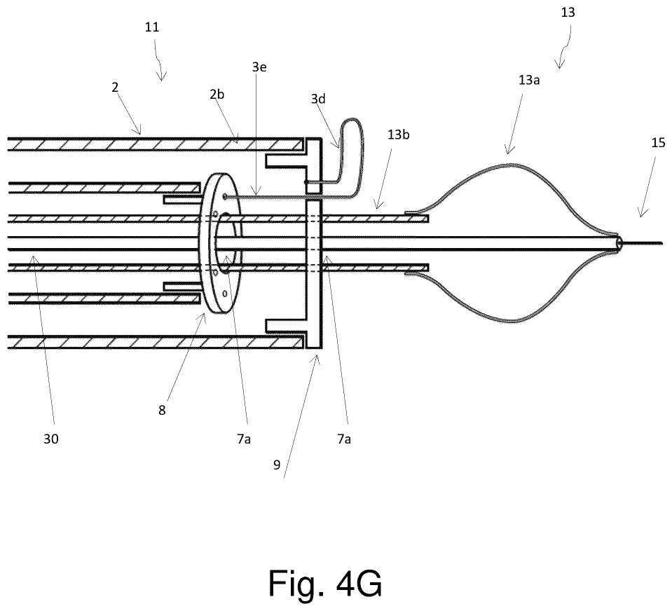

With reference to the different embodiments of FIG. 4, the catheter shaft 11 comprises an external tubular body 2 in which at least one control means 4 is included. In an embodiment, the control mean is one or more than one wire, or a tube. In an embodiment, the control mean is a hollow tube.

In an embodiment, the at least one control mean 4 is a wire and it is enveloped in a cover sheath 6. Preferably, when the control mean is more than one wire, each wire is housed individually in a cover sheath.

According to the art, each of the ablation elements or petals 3a is connected, via the fixed leg 3d, to the fixing point 14 and, via the movable leg 3e, to the control handpiece. In this embodiment, when the ablation head consists of 4 petals 3a, four legs, i.e., four wires, reach the control handpiece.

In a first embodiment according to the invention, two elements, a movable element 8 and a fixing element 9, are comprised in the catheter. The fixing element 9 is jointly connected to the catheter shaft 11. Each one of the petals 3a is connected, via the fixed leg 3d, to the fixing element 9 and, via the movable leg 3e, to the movable element 8. The movement of the movable element 8 is controlled from the control handpiece. In one embodiment, the movable element 8 is in the control handpiece. In this embodiment, one or more control means 4 reach the control handpiece from the movable element 8 which is in the catheter shaft.

The elements 8 and 9 are a cluster of multiple legs, wherein the movable element 8 is a cluster of movable legs, the fixing element 9 is a cluster of fixed legs. As an example, they are legs twisted together, or glued.

In a preferred embodiment, the element 8 are 9 are a physical element, as an example a disk, and the legs are attached to the physical element, the movable legs 3e to the movable element 8, the fixed legs 3d to the fixing element 9.

In an embodiment, the control mean is a control wire 4a. In an embodiment, the control means are two control wires 4a. In an embodiment, the control mean is a hollow tube 4b.

In this embodiment, regardless of the number of petals on the ablation head, only the control mean reaches the control handpiece, therefore solving the problem to increase the flexibility of the proximal portion of the catheter, wherein the problem is solved by reducing the number of wires comprised in the proximal portion of the catheter.

In a preferred embodiment, embodiment 1A, with reference to FIG. 4A, the fixing element 9 is a disk comprising a central hole. The fixing element 9 is fixed to the external tubular body 2 of the catheter shaft at the fixing point 14. The movable element 8, more proximal to the ablation head 3 along the catheter shaft than the fixing element 9, is movable into the catheter shaft.

In an embodiment, the fixing element 9 comprises connecting element 2b, wherein the connecting element 2b are an elongated portion extending from the fixing element, to increase the surface of the disk entering into contact with the external tubular body 2, therefore facilitating the connection among the two. As an example, the fixing element 9 is glued to the external tubular body 2.

In the embodiment depicted in FIG. 4A, the movable element 8 is a disk, too and the center of the movable element 8 is connected a control mean 4, which is a wire. The wire reaches the control handpiece passing through the hole in the fixing element 9. Advantageously, the wire is inside a cover sheath 6.

In embodiment 1B, with reference to FIG. 4B, the fixing element 9 is more proximal to the ablation head 3 than the movable element 8. The fixing element 9 is fixed to the control shaft. In the center of the movable element 8 is connected the control mean 4. In this embodiment, an internal tubular body 2a is inserted into the catheter shaft. The internal tubular body 2a is connected to the fixing element 9 via the connecting elements 2b and to the external tubular body 2 of the catheter shaft 11. The internal tubular body 2a elongates toward the control handpiece for a portion of a length enough to facilitate the movement of the movable element 8 which is conveniently inserted into the same.

In embodiments 1A and 1B, each one of the petals 3a is connected, via the legs 3c, to the elements 8 and 9. More in details, each one of the petals is connected via the fixed leg 3d to the fixing element 9, via the movable leg 3e to the movable element 8. In this manner, one of the legs 3c of the petal 3a is fixed, the other one is movable, thus the opening and closure of each one of the ablation petals 3 is controlled by the leg 3e, i.e., by the leg 3e movable via the control mean 4, to which it is connected via the movable element 8.

Conveniently, with reference to FIG. 4C, which is a top view of an embodiment of the fixing element 9, the fixing element 9 is a disk comprising a central hole 7a and, on the crown, through little holes 7c through which legs 3e pass, fixing points 10, to which legs 3d are fixed, and electric holes 7d for the passage of the electric cable. In an embodiment, where the ablator comprises four petals, the fixing element 9 comprises 4 fixing point 10, to fix the four legs 3d. However, it should be understood that in the fixing element 9 the number of holes 7 and of fixing points 10 could vary in the different embodiments.

Conveniently, with reference to FIG. 4F, which is a top view of an embodiment of the movable element 8, the movable element 8 is a disk comprising a central hole 7a and, on the crown, fixing points 10, to which legs 3e are fixed, and electric holes 7d for the passage of the electric cable. Moreover, rail holes 7b, for housing rails, when present, could be present on the crown. Preferably, the rail holes 7b are two and are positioned on the solid crown which constitutes the movable element B diametrically opposite each other.

In embodiment 1C, with reference to FIG. 4D, the fixing element 9 is more proximal to the ablation head 3 than the movable element 8. Both the elements 8 and 9 comprise a central hole 7a. The fixing element 9 is connected to the internal tubular body 2a, which is connected to the external tubular body 2 of the catheter shaft 11. The internal tubular body 2a extends from the fixing element 9 toward the proximal portion of the catheter shaft, for a small portion of the same, i.e., it extends to cover the length that the movable element 8 can cover in its movement. The fixing element 9 is jointly connected to the external tubular body 2, at the fixing point 14. In an embodiment, the diameter of the solid circle that constitutes the fixing element 9 is equal or greater than the diameter of the internal tubular body 2a, and this feature keeps the fixing element 9 connected to the shaft. The movable element 8 is free to move into the internal tubular body 2a.

In an embodiment, from the fixing element 9, toward the movable element 8, protrudes at least two tracks or rail 5. Each one of the rails 5 reach the movable element 8 and pass through one of the rail holes 7b.

Control means 4 are connected to the movable element 8, in this embodiment the control means is a hollow tube 4b.

In this embodiment, each of the ablation elements or petals 3a is connected, with the leg 3e, to the movable element 8, with the leg 3d, to the fixing element 9, so that one end is fixed, the other one is movable. Each one of the legs 3d passes through the through little hole 7c opened on the crown of the fixing element 9.

In each one of the embodiments 1A-C, the control means 4 are movable from the control handpiece, controlling the movement of the movable element 8. Given that the movable legs 3d of each one of the ablation elements 3a are jointly connected to the movable element 8, the result is that all the petals 3a are controlled by the control means 4. This implies that each one of the petals 3a opens and/or closes simultaneously.

In embodiment 1D, with reference to FIG. 4E, the fixing element 9 is more proximal to the ablation head 3 than the movable element 8. Both the elements 8 and 9 comprise a central hole 7a. The fixing element 9 is connected to the internal tubular body 2a. The internal tubular body 2a extends from the fixing element 9 toward the proximal portion of the catheter shaft, for a small portion of the same, i.e., it extends to cover the length that the movable element 8 can cover in its movement. The fixing element 9 is jointly connected to the external tubular body 2 at fixing point 14, via connecting elements 2b. The movable element 8 is free to move into the internal tubular body 2a. On the movable element 8 there are, in addition to the central hole 7a,
at least two rail holes 7b. Preferably, the additional holes 7b are two and are positioned on the solid crown which constitutes the movable element 8 diametrically opposite each other. From the fixing element 9, toward the movable element 8, protrudes at least two tracks or rail 5. Each one of the rails 5 reach the movable element 8 and pass through one of the additional holes 7b.

Two control wires 4a are connected to the movable element 8, in a diametrically opposite position, along the circumference of the same. The two control wires protrude along the tubular body to then merge into a single control wire before reaching the control handpiece.

In this embodiment, similarly to embodiment 1C, each of the ablation elements or petals 3a is connected, at the end 3e, to the movable element 8, at the end 3d, to the fixing element 9, so that one end is fixed, the other one is movable. Each one of the legs 3d passes through a through little hole 7c opened on the crown of the fixing element 9. In fact, in this embodiment, on the crown of the fixing element 9 there are through little holes 7c, wherein each one of the holes 7c houses a movable legs 3d.

Two diametrically opposite control wires 4a, or a control mean which is a hollow tube 4b, guarantee a correct IN and OUT movement of the movable element 8, keeping the same parallel to the fixing element 9 and longitudinal within the catheter shaft.

The above illustrated are exemplificative embodiments of the connection of an ablation head to the control handpiece, wherein the advantage is to have a flexible catheter, thanks to the control means solution. The catheter shaft could conveniently house, in addition to the ablation head, an anchor, wherein the anchor is in turn operatively connected to the control handpiece. In FIG. 4E embodiment, an anchor shaft 13b is depicted.

FIG. 4G is an exemplificative embodiment of a catheter shaft comprising an ablation head and an anchor.

The anchor 13 comprises anchor arms 13a and an anchor shaft 13b. The anchor shaft 13b passes through the central hole 7a of the fixing element 9 and through the central hole 7a of the movable element 8, to reach the control handpiece.

Inside the anchor shaft 13b, is comprised a guide wire lumen 30, into which a guide wire passes to reach the control hand piece. The guide wire, reaching the tip of the anchor, controls the opening and closure of anchor arms.

The person skilled in the art understands the here proposed combination of ablation head and anchor to be controlled at the control handpiece is possible in any of the above illustrated combination of fixing element and movable element, wherein the anchor shaft can reach the control handpiece, and this is possible when the fixing element comprise the central hole 7a and the movable element comprises the central hole 7a.

In a second embodiment, the present invention solves the problem to conform the Circumferential Linear Array of electrodes (CLA) to uneven surface.

Two features have been implemented, surprisingly allowing, alone or advantageously in a synergistic way, to conform the CLA of electrodes to uneven surface: i) a flexible distal portion of the catheter shaft; ii) each petal 3a free to move independently from the other petals comprised in the ablation head 3.

In an embodiment, each one of the petals 3a comprises:
a CLA 3b extending over a circumferential peripheral portion of the petal;
two legs 3c, each connected to an end of the CLA, each leg consisting in a curved section and a rail, the connection with the CLA being in correspondence with the curved section, the rail connecting the petals to the elements 8, 9.

When in the rest position, the legs and, partially, the CLA, are housed in the tubular body 2. When in operating position, FIG. 5A, the petals 3a protrude from the tubular body 2, exposing the CLA, the curved sections and, partially, the rails. This means that the legs 3c, when in operating position, comprises an exposed portion 3g of length D and an enveloped portion 3f, of length L.

Conveniently, the petals 3a, are made in a superelastic metal, i.e., a metal having the ability to undergo large deformations and immediately return to its undeformed shape upon removal of the external load. In an embodiment, the metal is Nitinol. The petals when reach the operating position, reassume the curvature previously imprinted to them via bending.

With reference to FIG. 5B, embodiment 2A, the two adjacent legs 3c of two adjacent petals 3a are housed in a single cover sheath 6. As an example, in an embodiment with four petals, there are four cover sheaths 6, each cover sheaths housing two legs 3c, belonging to two adjacent petals 3a.

In operating position, showed in FIG. 5B, the CLA and, partially, the side portions are exposed. In rest position, a portion of the CLA remains exposed, the remaining portion being housed in the cover sheath.

In embodiment 2B, with reference to FIG. 5C, each one of the legs 3c is housed in an independent cover sheath 6. In this embodiment, eight cover sheaths are needed for four petals. This embodiment allows each CLA 3b to move freely, being its movement independent of that of the adjacent petal, therefore each CLA is free to adapt to the surface.

In embodiment 2C, with reference to FIG. 5D, the cover sheaths 6, that in embodiments 2A, 2B were linear, are Y shaped. In this embodiment, each cover sheath 6 contains two legs, belonging to two adjacent petals.

In embodiment 2D, with reference to FIG. 5E, the ablation head comprises more than 4 petals 3a, in an embodiment 5, or 6, or 6, or 8 petals are comprised. Each leg is enveloped in an independent a cover sheath 6, or two legs belonging to two adjacent petals are enveloped together in a single cover sheath 6, the cover sheath being linear or Y shaped. In this embodiment, is the numerousness of the petals making the ablator capable to conform to uneven surface, wherein each one of the CLA 3b is shorter, and therefore more adaptable.

The curvature imprinted on the petals derives from the bending of the wire. The authors defined a bending capable to originate an advantageous curvature of the petals. The bending angles of ABCD can be comprised between 0 and 180°, wherein A, B, C, D are identified on FIG. 5F-I and are the proximal and the two distal angles on the petals, respectively. Advantageously, according to FIG. 5I, angles A, B, C and D are 90°. Varying the bending of the wire, different CLA geometry are obtained. As an example, CLA is a circle, or it is a square.

In an embodiment, with reference to FIG. 5G, H, angle D is 180°, angle C is 0°, A and B are about 90°

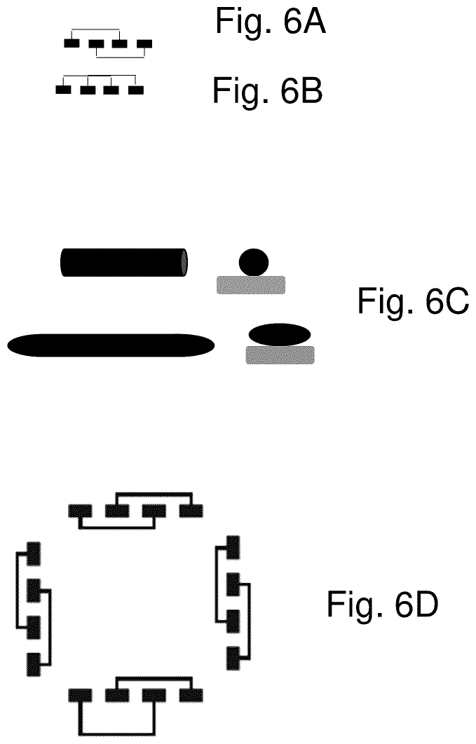

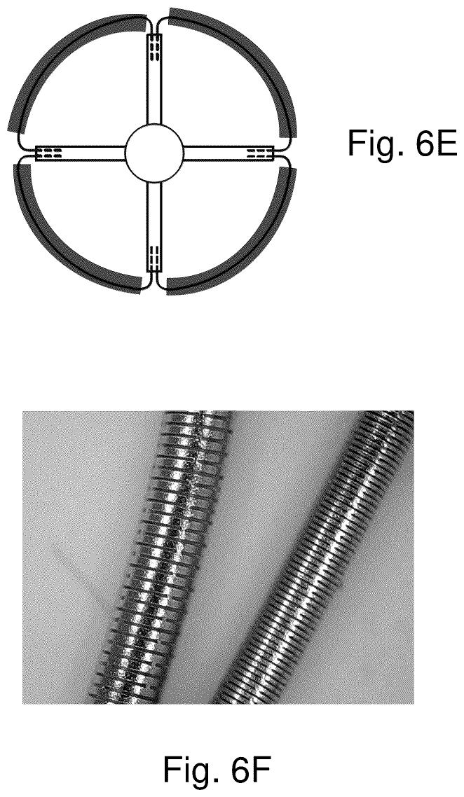

The portion 3b of the petals 3a has ablative electrodes. These electrodes could be linear (FIG. 6E) or point electrodes (FIG. 6A, B, D) connected in a certain way.

In an embodiment, a multiplicity of point electrodes is distributed on the petals. As an example, an electrode is formed by 2 or 3 or 4 or 5 or 6 or 8 electrodes. As an example, an electrode is formed by 4 electrodes. In this embodiment, according to FIG. 6A, representative of the point electrodes on a petal, the four electrodes on the same petal are connected by the same wire, thus simulating the effect of a linear electrode.

Connecting the point electrodes among them in different manner, allows the generation of different electrical field. In an embodiment, according to FIG. 6B, the four electrodes on a petal are connected two by two, i.e., the first connected to the third, the second to the fourth. This means that, having four electrodes connected in this manner on the four petals, is like having 8 ablative electrodes.

The generator has a control apparatus that connects and/or activates the electrodes independently, in different combinations.

In an embodiment, where the ablation head comprises 4 petals, each one of the petals comprise 4 electrodes. Each point electrode is 2 mm, spaced 3 mm from each other. The point electrodes are connected internally with electrical wire to form one electrode. This is schematically depicted in FIG. 6D.

Once in operating condition, the electrodes according to the present invention are perpendicular to the catheter shaft, being positioned on the portion 3b of the petal 3a.

To improve the contact between electrodes and tissue to be ablated, the cross section of the electrodes could be modified to maximise the surface in contact with the tissue. FIG. 6C illustrates two different electrode shapes, wherein electrodes have a circular (top) or an oval (bottom) cross section. When the cross section is oval, there is a better contact with the tissue, represented in gray. These shapes apply to linear or point electrodes.

In a third embodiment, a flexible linear electrode is provided. The electrode, defined a conforming electrode, has a plurality of flexible metal tubes each having a longitudinal axis, a proximal end, and a distal end. At least one helical cut is made through the wall of at least one of the flexible tubes along its longitudinal axis. The helical cuts are made by a laser. Alternatively, the helical cuts are made by mechanical cutting. Representative images of the flexible electrodes are in FIG. 6F.

Electrodes could be plated to improve the electrical conductivity to the tissue. As an example, platinum, or gold, or other biocompatible materials known by the person skilled in the art should be used to plate them.

In a fourth embodiment, a solution is provided to insulate the electrode from the petal structure, to keep the electrodes operatively independent from each other and to prevent energising the petal structure, which is connected to the control piece, in contact with the user. Moreover, formation of blood clot under the electrode must be prevented. Another problem related to the exposure of the electrodes is related to the point of connection between the electrode and the electric wire. In fact, waveform traveling through the electric wire and arriving to the connection point, in contact with blood could cause electrical spark at this point, due to high current density.

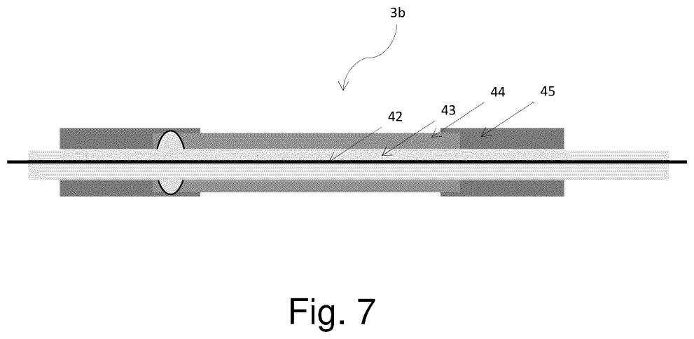

In one embodiment, depicted in FIG. 7, showing the portion 3b of a petal, the nitinol wire 42 is covered with a polyimide tube 43. The electrode 44 is put on the polyimide tube 43. Both side of the electrode are blocked to prevent blood flowing and clot under the electrodes. This is obtained by gluing the electrodes, or by using cover tube 45, as an example polyurethane or PEBAX cover tubes.

In a fifth embodiment, the present invention allows stamping ablation, wherein stamping ablation technique consists of applying ablation energy to heart surfaces that are continuous. This technique is useful for example to do ablation on the walls of the heart that does not require anchoring, for example, in Posterior Wall Isolation.

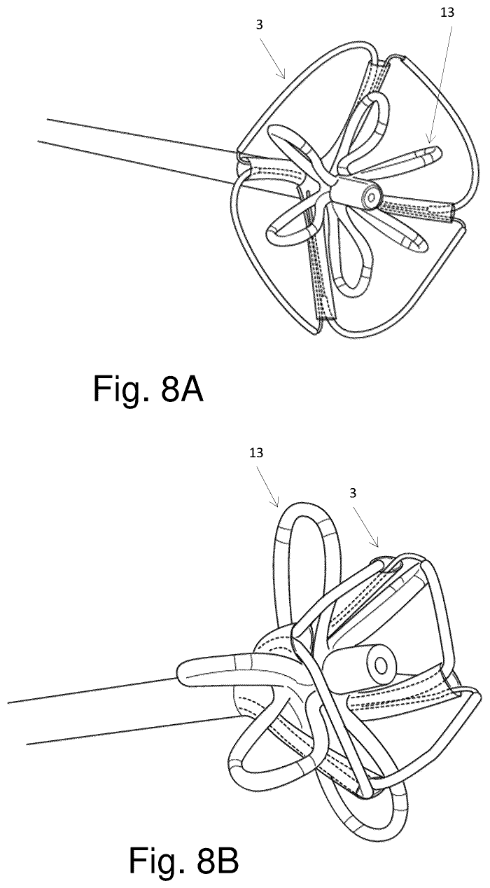

FIG. 8A, B, describes a reciprocal position of the ablation head 3 which is an umbrella ablation head and the anchor 13, making the ablator working in a “stamping” fashion.

The anchor on the catheter of the present invention has the capability to collapse into the ablation head 3 allowing the CLA ablative surface to be in front of the anchor itself, without the anchor interfering with the tissue.

Once the anchor is collapsed, the user can adjust the CLA size to define the stamping ablation size. A method to ablate continues heart tissue is claimed. The method comprising:
making available a catheter apparatus according to the present invention:
collapsing the anchor into the ablation head;
adjusting the size of the ablation head, by adjusting the petals size;
advancing the ablation head in the stamping position toward the heart continuous tissues; and
applying energy; and repeating the operation as needed.

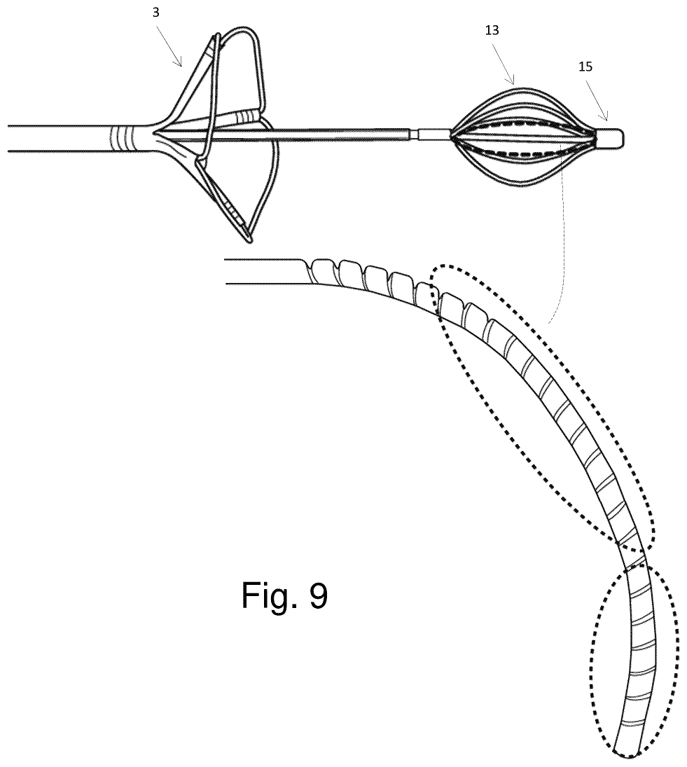

A further problem to be faced when using flexible anchor structures is a kinking of the guide wire lumen 40 when the anchor is pushed against the tissue, which cause kinking of the tube that will be difficult to recover, or to straight. In a sixth embodiment, the pushability and the flexibility of the guide wire lumen is improved. A flexible metal tube is used as a guide wire 40 for the anchor. Reference is made to FIG. 9. The tube is laser cut with different pattern to allow multiple level of flexibility for each section of the tube. As shown in the enlarged view, in an embodiment the guide wire 40 comprises a more flexible section, to allow an easy deflection of the same, which is proximal to the ablator head 3 and a less flexible section, to prevent anchor from kinking, more proximal to the catheter tip 15.

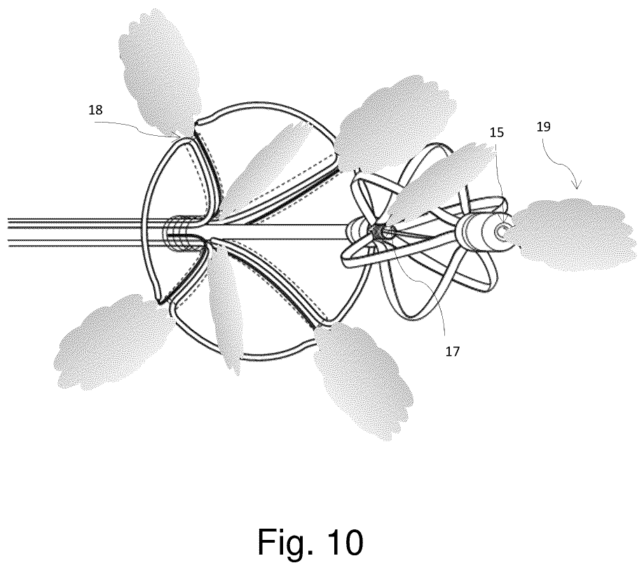

In a seventh embodiment, any empty spaces in the catheter in contact with the blood need to be flushed with saline before being inserted into the patient blood vessel, to avoid transferring air molecules trapped into these empty spaces into the blood circulation. Another reason for the capability to flush these spaces is to prevent the blood clot. Reference is made to FIG. 10, the clouds are indicative of the flush 19 exiting from the CLA flushing port 18, from the shaft, from the anchor flushing port 17, and from the tip. These can be achieved by one or more port located on the control handpiece.

### REFERENCE NUMBERS

1. catheter apparatus
2. external tubular body
   2a. internal tubular body
   2b. connecting elements
3. ablation head
   3a. ablation element or petals;
   3b. circumferential peripheral portion
   3c. legs
   3d. fixed leg
   3e. movable leg
   3f. legs, enclosed portion
   3g. legs, exposed portion
4. control means
   4a. control wire
   4b. hollow tube
5. tracks or rail
6. cover sheath
7. holes
   7a. central hole
   7b. rail holes
   7c. through little holes
   7d. electric holes
8. movable element
9. fixing element
11. catheter shaft
12. control handpiece
13. Anchor
   13a. anchor arms
   13b. anchor shaft
14. fixing point
15. catheter tip
16. deflectable sheath
17. anchor flushing port
18. CLA flushing port
19. flush
20. Generator
21. Foot switch
22. Graphical user interface
23. ECG processing unit
24. ECG interface box
25. Interface unit (catheter/generator)
26. Interface
31. Dispersive pad
32. Surface ECG electrodes
33. Subject
40. Guide wire
41. Guide wire lumen
42. Nitinol wire
43. Polyimide tube
44. Electrode
45. Cover tube
99. Right atrium
100. left atrium
101. a-d pulmonary veins
102. septum

What is claimed is:

1. A catheter apparatus, comprising
a control handpiece,
a catheter shaft,
an ablation head,
a catheter tip,
said catheter shaft having an external tubular body and comprising two portions: a proximal portion, closer to the control handpiece, and a distal portion, toward the catheter tip, wherein along said catheter shaft a fixing point is defined, wherein said fixing point is in said distal portion of said catheter shaft, wherein said ablation head comprises at least two ablation elements or petals, each petals comprising:
a circumferential peripheral portion, substantially along an arc of circumference having the longitudinal axis of the catheter shaft as its centre, comprising one or more electrodes, thus forming a circumferential linear ablation array (CLA);
two legs, each one connected to an end of the portion, in correspondence with a curved section therein, wherein one legs is a fixed leg, the other one is a movable leg;
wherein two elements, a movable element and a fixing element, are comprised in said catheter, the movable element being movable along the catheter shaft, the fixing element being connected to the catheter shaft, at said fixing point;
wherein each one of the petals is connected, via said fixed leg, to said fixing element and, via said movable leg, to said movable element;
characterized in that
one or more control means, selected in the group consisting of a control wire or a hollow tube, from said movable element reach the control handpiece, said control means being in a number which is equal or inferior to the number of the petals comprised on said ablation head; the opening and closure of said petals being controlled via said movable element form said control handpiece.

2. The catheter according to claim 1, wherein said movable elements and fixing elements are physical elements and said legs are attached to said physical element, said movable legs to said movable element, said fixed legs to said fixing element.

3. The catheter according to claim 1, wherein said fixing element comprises connecting elements, wherein said connecting elements are an elongated portion extending from said fixing element, to increase the surface of said disk entering into contact with said external tubular body, therefore facilitating the connection among the two.

4. The catheter shaft according to claim 1, wherein said fixing element is more proximal to the ablation head than said movable element.

5. The catheter shaft according to claim 4, wherein said fixing element is fixed to the control shaft and, in the centre of the movable element, is connected to the control mean.

6. The catheter shaft according to claim 5, wherein an internal tubular body is inserted into the catheter shaft, said internal tubular body elongating toward the control handpiece for a portion of a length enough to facilitate the movement of the movable element which is housed into the same.

7. The catheter according to claim 1, wherein said movable and fixing elements are, independently one from the other, a cluster of multiple legs, wherein the movable element is a cluster of movable legs, the fixing element is a cluster of fixed legs.

8. The catheter according to claim 1, wherein said fixing element is a disk comprising a central hole, said fixing element being fixed to the external tubular body of the catheter shaft at the fixing point and said movable element, more proximal to said ablation head along said catheter shaft than said fixing element, is movable into the catheter shaft.
